(19) United States
(12) Patent Application Publication
(10) Pub. No.: US 2024/0255458 A1
(43) Pub. Date: Aug. 1, 2024

(54) SYSTEMS, METHODS, AND MEDIA FOR PREDICTING PRESENCE OF ONE OR MORE MOLECULES USING CHEMICAL SENSORS

(71) Applicant: NUtech Ventures, Lincoln, NE (US)

(72) Inventors: Eric MARKVICKA, Lincoln, NE (US); Jennifer YENTES, College Station, TX (US); Stephen RENNARD, Omaha, NE (US); Jason FINNEGAN, Lincoln, NE (US)

(21) Appl. No.: 18/692,261

(22) PCT Filed: Sep. 14, 2022
(86) PCT No.: PCT/US2022/043471

§ 371 Date: Mar. 14, 2024

Related U.S. Application Data
(60) Provisional application No. 63/243,974, filed on Sep. 14, 2021.

(51) Int. Cl. G01N 27/22; G16H 40/67

(52) U.S. Cl. CPC G01N 27/22; G16H 40/67; G01N 2800/50

(57) ABSTRACT

In accordance with some embodiments of the disclosed subject matter, mechanisms (which can, for example, include systems, apparatuses, methods, and media) for predicting a physiological state of a subject using one or more chemical sensors. In some embodiments, a system comprises: a plurality of chemical sensors; a transmitter; and a processor coupled to the plurality of chemical sensors and the transmitter, the processor programmed to: measure, for each of the plurality of sensors, a respective value of a plurality of values; and transmit, via the transmitter, a profile based on the plurality of values to a computing device.

### CROSS-REFERENCE TO RELATED APPLICATIONS

This application is based on, claims the benefit of, and claims priority to U.S. Provisional Application No. 63/243,974, filed Sep. 14, 2021, which is hereby incorporated herein by reference in its entirety for all purposes.

### STATEMENT REGARDING FEDERALLY SPONSORED RESEARCH

This invention was made with government support under U54 GM115458 awarded by the National Institutes of Health. The government has certain rights in the invention.

### BACKGROUND

Current diagnostic tests for many conditions involve invasive and somewhat uncomfortable upper respiratory tract swabs that depend on the skills of the person taking the sample and timing of the sample collection. For example, the inability to deploy adequate screening and diagnostic testing has seriously compromised the response to the COVID pandemic in the United States. Screening tests currently used include symptom questionnaires and thermometers. However, at least 40% of individuals infected with SARS-CoV-2 are asymptomatic and those who develop symptoms are infectious in the pre-symptomatic phase. As a consequence, current screening detects 30% or less of infected individuals. The low sensitivity of screening tests has compromised the effectiveness of mitigation strategies. While polymerase chain reaction (PCR) and antigen tests have much higher sensitivity, these tests are often relatively costly and/or time consuming.

Accordingly, new systems, methods, and media for predicting presence of one or more molecules using one or more chemical sensors are desirable.

### SUMMARY

In accordance with some embodiments of the disclosed subject matter, systems, methods, and media for predicting presence of one or more molecules using one or more chemical sensors are provided.

In accordance with some embodiments of the disclosed subject matter, a system for predicting presence of one or more airborne using one or more chemical sensors is provided, the system comprising: a plurality of chemical sensors; a transmitter; and a processor coupled to the plurality of chemical sensors and the transmitter, the processor programmed to: measure, for each of the plurality of sensors, a respective value of a plurality of values; and transmit, via the transmitter, a profile based on the plurality of values to a computing device.

In some embodiments, the transmitter comprises a radio-frequency antenna.

In some embodiments, the processor is further programmed to: transmit the profile via near-field communication.

In some embodiments, the processor is configured to receive power via the radio-frequency antenna.

In some embodiments, the processor is further programmed to: perform a principal component analysis based on the plurality of values.

In some embodiments, the processor is further programmed to: generate the profile based on results of the principal component analysis.

In some embodiments, the profile comprises results of the principal component analysis.

In some embodiments, the processor is further programmed to: predict a physiological state of a subject based on results of the principal components analysis; and generate the profile based on the predicted physiological state of the subject.

In some embodiments, the system further comprises a power source electronically coupled to the processor.

In some embodiments, the power source comprises a battery.

In some embodiments, each of the plurality of values is a conductance value.

In some embodiments, each of the plurality of values is a capacitance value.

In some embodiments, the physiological state is indicative of whether the subject is infected with SARS-CoV-2 virus.

In some embodiments, the plurality of sensors comprises a polymer-based sensor.

In some embodiments, the plurality of sensors comprises a metal-oxide-based sensor.

In accordance with some embodiments of the disclosed subject matter, a system for predicting a physiological state of a subject using one or more chemical sensors is provided, the system comprising: a processor programmed to: receive a profile based on a plurality of values generated by a sensor device comprising a plurality of chemical sensors, predict a physiological state of a subject based on the profile.

In some embodiments, the system comprises a server, and wherein the profile is received from the sensor device via a mobile computing device.

In some embodiments, the system comprises a server, and wherein the profile is received from the sensor device via a wide area network.

In some embodiments, the system comprises a radio-frequency antenna.

In some embodiments, the processor is further programmed to: receive the profile via near-field communication.

In some embodiments, the processor is configured to provide power to the sensor device via the radio-frequency antenna.

In some embodiments, the processor is further programmed to: predict a physiological state of a subject based on results of a principal components analysis of the plurality of values; and generate the profile based on the predicted physiological state of the subject.

In some embodiments, the processor is further programmed to: perform the principal component analysis based on the plurality of values.

In some embodiments, the profile is based on results of the principal component analysis.

In some embodiments, each of the plurality of values is a conductance value.

In some embodiments, each of the plurality of values is a capacitance value.

In some embodiments, the physiological state is indicative of whether the subject is infected with SARS-CoV-2 virus.

In some embodiments, the plurality of sensors comprises a polymer-based sensor.

In some embodiments, the plurality of sensors comprises a metal-oxide-based sensor.

In some embodiments, the processor is further programmed to: receive physiological data generated by a physiological sensor; and predict the physiological state of the subject based on the profile and the physiological data received from the physiological sensor.

In some embodiments, the processor is further programmed to: receive the physiological data from the sensor device.

In accordance with some embodiments of the disclosed subject matter, a system for predicting presence a physiological state of a subject is provided, the system comprising: a sensor device comprising: a plurality of chemical sensors, wherein the plurality of chemical sensors comprise polymer-based sensor or a metal-oxide-based sensor; a transmitter; and a processor coupled to the plurality of chemical sensors and the transmitter, the processor programmed to: measure, for each of the plurality of sensors, a respective value of a plurality of values, wherein each of the plurality of values is a conductance value or each of the plurality of values is a capacitance value; and transmit, via the transmitter, a profile based on the plurality of values to a computing device; and a computing device comprising: a processor programmed to: receive the profile; and predict a physiological state of a subject based on the profile, wherein the physiological state of the subject comprises: whether the subject is infected with SARS-CoV-2 virus; or whether the subject is likely to experience disease caused by Clostridioides difficile.

In accordance with some embodiments of the disclosed subject matter, a system for predicting presence of one or more environmental threats using one or more chemical sensors is provided, the system comprising: a processor programmed to: receive a profile based on a plurality of values generated by a sensor device comprising a plurality of chemical sensors; predict presence of one or more environmental threats based on the profile.

In some embodiments, the environmental threat is indicative of whether the sensor device has been exposed to one or more of: ammonia, chlorine, nitric acid, sulfur dioxide, carbon monoxide, isobutane, methane, ethanol, nitrous oxide, hydrogen sulfide, acetone, chloroform, benzene, toluene, ethyl alcohol, methyl alcohol, ethyl benzene, xylene, benzene, toluene, or gasoline.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for predicting of one or more molecules using one or more chemical sensors in accordance with some embodiments of the disclosed subject matter.

FIG. 2 shows an example of hardware that can be used to implement a chemical sensor, a mobile near field communication reader device, and a server, shown in FIG. 1 in accordance with some embodiments of the disclosed subject matter.

FIG. 3 shows an example of a device with multiple chemical sensors for predicting presence of one or more molecules in accordance with some embodiments of the disclosed subject matter.

FIG. 4 shows an example of another device with multiple chemical sensors for predicting presence of one or more molecules in accordance with some embodiments of the disclosed subject matter.

FIGS. 5A and 5B show examples of a chemical sensor that can be used to implement a device with one or more chemical sensors for predicting presence of one or more molecules in accordance with some embodiments of the disclosed subject matter.

FIG. 6 shows an example of a process for predicting presence of one or more molecules in accordance with some embodiments of the disclosed subject matter.

FIG. 7 shows examples of techniques for configuring a device with multiple chemical sensors for predicting presence of one or more molecules as a wearable device in accordance with some embodiments of the disclosed subject matter.

FIG. 8 shows an example of relative output of four different chemical sensors over time when exposed to a known concentration of methanol.

FIG. 9 shows an example of results of a principal component analysis (PCA) of outputs of the four different chemical sensors used to produce the example of FIG. 8 when analyzed at after exposure to a various volatile organic chemicals.

### DETAILED DESCRIPTION

In accordance with various embodiments, mechanisms (which can, for example, include systems, methods, and media) for predicting presence of one or more molecules using one or more chemical sensors are provided.

In accordance with some embodiments of the disclosed subject matter, mechanisms described herein can facilitate non-invasive prediction of a physiological state of a subject using one or more chemical sensors that can be used to detect the presence of chemicals, such as volatile organic compounds. For example, mechanisms described herein can be used to record readings from chemical sensors associated with an electronic-nose device in proximity of a subject, and predict the subjects physiological state based on the readings from the chemical sensors. In a more particular example, mechanisms described herein can be used to record readings from multiple chemical sensors associated with an electronic-nose device in proximity of a subject, and can predict whether the subject is infected with a particular pathogen (e.g., a virus such as SARS-CoV-2, a bacterium such as Clostridioides difficile (C. diff), etc.) and/or afflicted with a particular condition (e.g., cystic fibrosis, asthma, and chronic obstructive pulmonary disease) based on the readings from the chemical sensors.

In some embodiments, mechanisms described herein can be used to improve screening using relatively inexpensive, non-invasive wearable sensors that can provide relatively high quality data, which can accelerate the speed of diagnostic information and the potential accuracy of the information in making a diagnosis.

In some embodiments, mechanisms described herein can be used to implement a battery-free e-nose sensor device and/or a physiological state prediction system that utilizes data from an e-nose sensor. Such a sensor device and/or system can be configured to predict one or more physiological states. For example, a sensor device and/or system implemented in accordance with mechanisms described herein can be configured to screen for SARS-CoV-2 infection. In such an example, a battery-free, wearable electronic nose sensor device can be used to continuously monitor and detect volatile organic compounds (VOCs) that are associated with SARS-CoV-2 infection. In a more particular example, mechanisms described herein can be used to validate an e-nose sensor device, and can be used to implement a sensor output profile (sometimes referred to as a smellprint-signature) classification technique(s) for diagnosing patients admitted to the hospital with suspected SARS-CoV-2 infection. The COVID pandemic has highlighted the importance of rapid, readily available screeners and diagnostics to identify infected individuals. Portable electronic nose sensing devices can detect volatile organic compounds (VOCs). These compounds are end products of human metabolism and/or of enteric bacteria, and are excreted through the skin and/or exhaled breath. Sensing devices implemented using techniques described herein can be configured as wearable devices that can provide the capability of continuously monitoring a subject. As described above, mechanisms described herein can be used to implement an electronic nose sensor device (e.g., implemented as a wearable device, which may or may not be battery free, implemented as a non-wearable device, which may or may not be wall powered, etc.) to serve as a screener and detect VOC patterns associated with subjects infected with SARS-CoV-2, who may be symptomatic or asymptomatic for COVID-19, without being invasive. Note that although mechanisms described herein are often described in connection with predicting whether a subject is infected with SARS-CoV-2, this is an example, and mechanisms described herein can be used to predict other physiological states (e.g., experiencing disease caused by C. diff infection, cystic fibrosis, asthma, chronic obstructive pulmonary disease, etc.) and/or environmental threats (e.g., the presence of a particular chemical that is hazardous to humans, the presence of particular pollutants, etc.)

In some embodiments, mechanisms described herein can be used to implement a powered electronic nose sensor device (e.g., battery powered, wall powered) that can be used to monitor for chemicals in a subject's space (e.g., a subject or patient's room), and provide information that can be used to predict a physiological state and/or to make a diagnosis, as described above in connection with as the wearable sensor.

In some embodiments, a device and/or system implemented in accordance with mechanisms described herein can be used as a stand-alone device and/or service. Additionally or alternatively, in some embodiments, a device and/or system implemented in accordance with mechanisms described herein can be used in combination with one or more other physiological sensors to improve the accuracy (e.g., the specificity) of a screening system.

Improved identification of individuals who are likely infected with SARS-CoV-2 can facilitate isolation efforts and can help mitigate the impact of outbreaks when they occur. Relatively inexpensive sensor devices implemented in accordance with mechanisms described herein can be readily-deployable to provide a path towards deployment to the general population, which can permit individuals in high risk environments to be monitored continuously and/or at frequent intervals. This can allow earlier and more complete identification of infected individuals than current approaches. In some embodiments, mechanisms described herein can be used to implement a relatively inexpensive, at least partially automated, and at least semi-passive system that can be used to implement cost-effective screening for one or more physiological states and/or conditions.

C. diff infection (CDI) is the most important cause of health care-associated diarrhea. Despite efforts to limit antibiotic usage, the most common risk factor for CDI, there were 224,000 cases and 12,800 deaths in 2017 compared to 250,000 cases and 14,000 deaths in 2013. Importantly, many cases develop weeks after antibiotic exposure, often after a patient has been discharged from the hospital. Other causes of diarrhea and the fact that C. diff can be present in the bowels without causing disease further complicate diagnosis. At present, there are no definitive tests that can determine when or if clinically significant CDI is present or is likely to develop.

C. diff causes disease through the release of toxins that damage the lining of the large intestine. Some strains of C. diff do not produce toxins and do not cause disease. Individuals can become ill when they harbor a toxin producing strain and when the balance of bacteria in the gut is altered and leads to over-production of C. diff toxins and intestinal damage. Antibiotics are believed to cause to disease by altering the balance of gut bacteria.

Tests that can identify C. diff toxin are reliable and readily available. However, diagnosis of CDI that requires treatment remains difficult because many individuals are colonized with C. diff, but have diarrhea for other reasons. Importantly, up to 15% of the general population may be colonized with C. diff and have no evidence of disease. Treatment of these persons for CDI is not recommended.

Since there is no diagnostic test to determine if CDT is present and likely to cause disease, current practice is to wait until diarrhea develops and then determine if toxin producing C. diff is present. Since there may be other causes for diarrhea, a diagnosis of CDT then depends on an appropriate response to CDT treatment. This approach results in late treatment of patients with CDI and delayed diagnosis and treatment of patients with other causes of diarrhea.

As described above, tests are readily available that can identify the presence of C. diff and can identify if strains that have the potential to cause disease (e.g. toxin producing strains) are present. However, that information alone is insufficient to determine if a patient should be treated. A diagnostic approach that can recognize when C. diff has transitioned from a ‘colonizing’ bacterium to one causing disease is desirable. Current toxin assays are inadequate to do so. Severe diarrhea due to CDI may develop weeks after a course of antibiotics. An effective technique of monitoring for CDI that can detect disease before the onset of severe symptoms and that can be used over a time frame of months is desirable.

In some embodiments, mechanisms described herein can be used to monitor the effects of CDI that lead to disease. For example, C. diff that causes disease when toxins damage the intestinal wall. Detection of toxins (or the genes that produce them) alone can determine risk, but cannot distinguish colonization from disease. The intestinal damage, which is an important feature of disease caused by C. diff, leads to a variety of effects that may be measurable. For example, C. diff produces characteristic volatile organic compounds (VOCs), as do other gut bacteria. The absorption of such molecules is altered by intestinal damage, and such VOCs are excreted in exhaled breath and through the skin where they can be detected using a chemical sensor described herein.

In accordance with some embodiments of the disclosed subject matter, mechanisms described herein can detection of particular molecules in an environment (e.g., airborne molecules, liquids, etc.) using one or more chemical sensors that can be used to detect the presence of chemicals, such as volatile organic compounds. For example, mechanisms described herein can be used to record readings from chemical sensors associated with an electronic-nose device, and predict whether particular molecules are present in the environment based on the readings from the chemical sensors. In a more particular example, mechanisms described herein can be used to record readings from multiple chemical sensors associated with an electronic-nose device in an environment (e.g., worn by a subject, placed in a stationary location, etc.), and can predict whether a particular molecule(s) are present in the environment based on the readings from the chemical sensors.

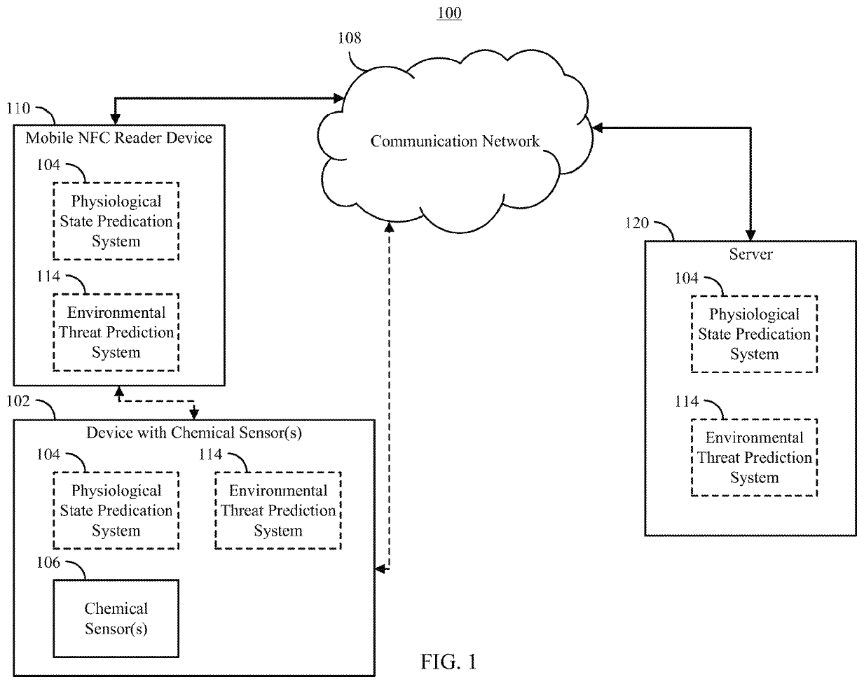

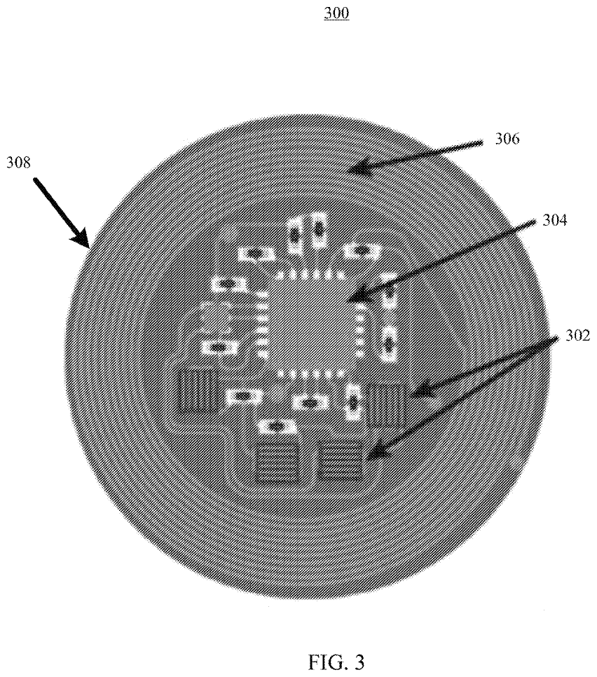

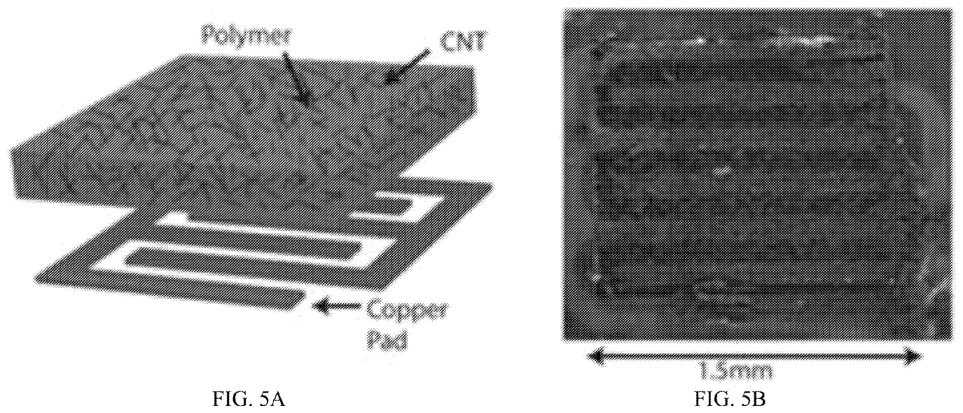

FIG. 1 shows an example of a system for predicting a physiological state of a subject using one or more chemical sensors in accordance with some embodiments of the disclosed subject matter. As shown in FIG. 1, a device 102 with one or more chemical sensors 106 can be any suitable device, such as a wearable device, a wall mounted device, etc. In some embodiments, chemical sensors 106 can be implemented using any suitable technique or combination of techniques, for example, as described below in connection with FIGS. 3, 5A, and 5B. In some embodiments, device 102 can execute at least a portion of a physiological state prediction system 104 to predict a physiological state of a subject based on output(s) of chemical sensor 106. Alternatively, in some embodiments, physiological state prediction system 104 can be entirely omitted from device 102. In some embodiments, device 102 can execute at least a portion of an environmental prediction system 114 to predict the presence of an environmental threat(s) based on output(s) of chemical sensor 106. Alternatively, in some embodiments, environmental prediction system 114 can be entirely omitted from device 102. In such embodiments, device 102 can capture data from chemical sensors 106, and transmit the data to another device for analysis (e.g., computing device 110, a server 120, etc.). In some embodiments, device 102 can be configured to communicate locally with a computing device 110 (e.g., a mobile near-field communication reader device). For example, device 102 can be incorporated with computing device 110 (e.g., device 102 can be configured as part of a device for capturing, storing, communicating, and/or analyzing physiological data). As another example, device 102 can be connected to computing device 110 by a cable, a direct wireless link, etc. Additionally or alternatively, in some embodiments, device 102 can be located locally and/or remotely from computing device 110, and can communicate data to computing device 110 (and/or server 120) via a communication network (e.g., communication network 108).

In some embodiments, computing device 110 (e.g., a mobile near-field communication reader device) can receive multiple data from device 102. In some embodiments, computing device 110 can execute at least a portion of physiological state prediction system 104 to predict a physiological state of a subject based on output(s) of chemical sensor 106 and/or at least a portion of environmental prediction system 114 to predict the presence of an environmental threat(s) based on output(s) of chemical sensor 106.

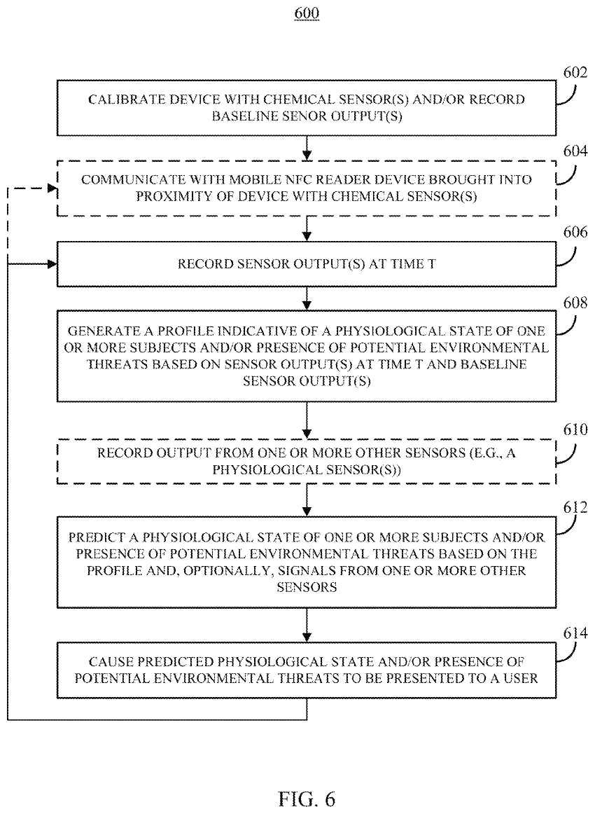

Additionally or alternatively, in some embodiments, device 102 and/or computing device 110 can communicate data captured and/or generated by device 102 (and/or computing device 110) to server 120 over a communication network 108, and server 120 can execute at least a portion of physiological state prediction system 104 and/or at least a portion of environmental prediction system 114. In such embodiments, server 120 can return information to computing device 110 (and/or any other suitable computing device) indicative of an output of physiological state prediction system 104 and/or indicative of an output of environmental prediction system 114. In some embodiments, physiological state prediction system 104 can execute one or more portions of process 600 described below in connection with FIG. 6. Additionally or alternatively, in some embodiments, environmental prediction system 114 can execute one or more portions of process 600 described below in connection with FIG. 6.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, etc.

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

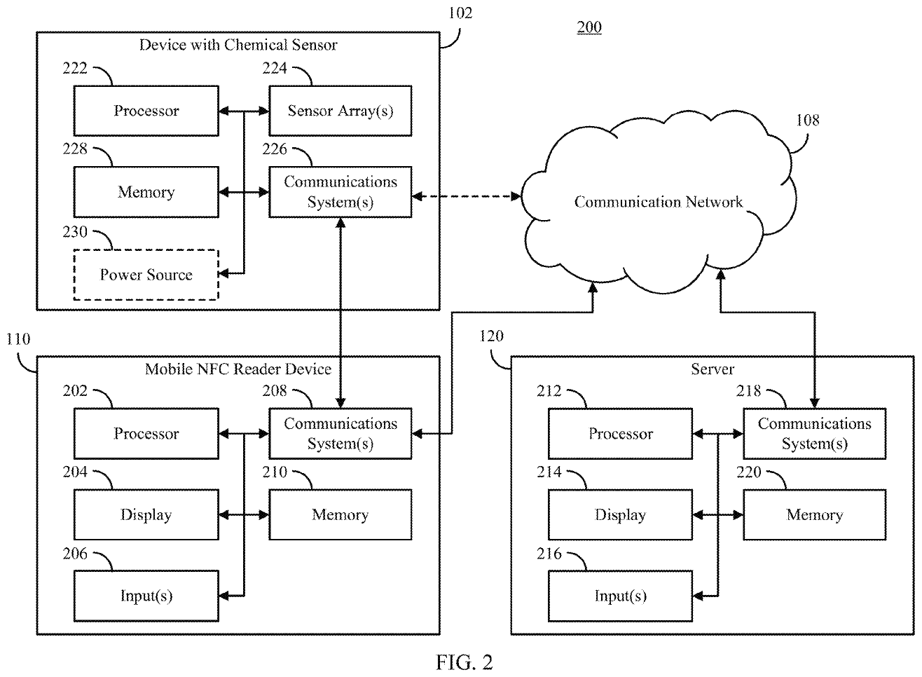

FIG. 2 shows an example of hardware that can be used to implement a device with chemical sensors 102, a mobile near field communication reader device 110, and a server 120, shown in FIG. 1 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), an application specific integrated circuit (ASIC), a field-programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a near-field communication (NFC) connection, a radio-frequency identification (RFID) connection, a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with one or more sensor devices 102 via communications system(s) 208, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to receive raw and/or processed data from sensor device 102, record sensor output data, generate a profile indicative of a physiological state of a subject, transmit raw and/or processed data indicative of outputs of sensor device 102 (e.g., to server 120), receive and/or record data from one or more other physiological sensors, predict a physiological state of a subject, cause a predicted physiological state to a user, receive data and/or content from server 120, transmit data and/or other information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, an NFC connection, an RFID connection, etc.

In some embodiments, memory 220 can include any suitable storage device or devices that can be used to store instructions, values, etc., that can be used, for example, by processor 212 to present content using display 214, to communicate with one or more computing devices 110, to communicate with one or more sensor devices 102, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to receive raw and/or processed data from sensor device 102, receive raw and/or processed data from computing device 110, record sensor output data, generate a profile indicative of a physiological state of a subject, receive and/or record data from one or more other physiological sensors, predict a physiological state of a subject, cause a predicted physiological state to a user, receive data and/or content from server 120, transmit data and/or other information to server 120, etc.

In some embodiments, sensor device 102 can include a processor 222, a sensor array 224 (e.g., including one or more sensors) or multiple sensor arrays 224, one or more communications systems 226, memory 228, and/or a power supply 230. In some embodiments, processor 222 can be any suitable hardware processor or combination of processors, such as a CPU, a GPU, an ASIC, an FPGA, etc. In some embodiments, sensor array 224 can include be any suitable components to generate data indicative of the presence of one or more chemicals (e.g., airborne chemicals). For example, sensor array 224 can include one or more polymer-based sensors that can change properties (e.g., resistance, dielectric permittivity) in response to absorbing chemicals (e.g., VOCs) that come into contact with the sensor (e.g., as described below in connection with FIGS. 3 and 5). As another example, sensor 224 can include one or more metal-oxide-based sensors that can change properties (e.g., resistance) in response to absorbing chemicals (e.g., VOCs) that come into contact with the sensor.

In some embodiments, communications systems 226 can include any suitable hardware, firmware, and/or software for communicating information to computing device 110 (and, in some embodiments, over communication network 108 and/or any other suitable communication networks). For example, communications systems 226 can include one or more transceivers, one or more communication chips and/or chip sets, etc. In a more particular example, communications systems 226 can include hardware, firmware and/or software that can be used to establish an NFC connection, an RFID connection, a 315 megahertz (MHz) connection, a 434 MHz connection, an XBee connection (e.g., based on the IEEE 802.15.4 standard), a LoRa connection, a Wi-Fi connection, a Bluetooth connection, a cellular connection, an Ethernet connection, a wired connection using any suitable port and/or communication standard (e.g., USB, RS-232, power line communication (PLC), etc.), etc.

Note that, although not shown, sensor device 102 can include any suitable inputs and/or outputs. For example, sensor device 102 can include input devices and/or sensors that can be used to receive user input, such as a keyboard, a mouse, a touchscreen, a microphone, a trackpad, a trackball, hardware buttons, software buttons, etc. As another example, sensor device 102 can include any suitable display devices, such as a computer monitor, a touchscreen, a television, etc., one or more speakers, etc.

In some embodiments, memory 228 can include any suitable storage device or devices that can be used to store instructions, values, raw sensor data, processed sensor data, etc., that can be used, for example, by processor 222 to: read data from sensor array 224; cause data to be stored in memory 228; cause data to be transmitted to a computing device (e.g., computing device 110) and/or server (e.g., server 120); communicate with one or more computing devices 110; etc. Memory 228 can include any suitable volatile memory, non-volatile memory, storage, or any suitable combination thereof. For example, memory 228 can include RAM, ROM, EEPROM, one or more flash drives, one or more hard disks, one or more solid state drives, one or more optical drives, etc. In some embodiments, memory 228 can have encoded thereon a program for controlling operation of sensor device 102. In such embodiments, processor 222 can execute at least a portion of the program to generate raw sensor data (e.g., by reading one or more sensors), to generate processed sensor data (e.g., by performing one or more operations to generate processed sensor data based on raw sensor data), transmit information and/or content (e.g., raw sensor data, processes sensor data) to one or more computing devices 110, receive information and/or content from one or more computing devices 110, transmit information and/or content (e.g., raw sensor data, processes sensor data) to one or more servers 120, receive information and/or content from one or more servers 120, receive instructions from one or more devices (e.g., a personal computer, a laptop computer, a tablet computer, a smartphone, etc.), etc. In some embodiments, memory 228 can be omitted. For example, sensor device 102 can be implemented using circuitry (e.g., circuitry of an ASIC, circuitry of an FPGA, analog circuitry including one or more comparators, etc.) configured to perform functions of sensor device 102 described herein.

In some embodiments, power source 230 can be implemented using any suitable technique or combination of techniques. For example, power source 230 can be implemented using a battery. As another example, power source 230 can be implemented using a connection to a power grid (e.g., 110 volt power supplied via a wall outlet). In some embodiments, power source 230 can be omitted. For example, in some embodiments, power can be supplied to sensor device 102 via NFC (or any other suitable protocol configured to remotely provide power), and sensor device 102 can be unpowered when not in communication with a device capable of providing power via NFC.

FIG. 3 shows an example of a device 300 with multiple chemical sensors for predicting a physiological state of a subject in accordance with some embodiments of the disclosed subject matter. In some embodiments, device 300 can include sensors 302, a processor 304, an antenna 306, and various other components (not labeled) that facilitate readout of data from sensors 302 and transmission of data using antenna 306. In some embodiments, any suitable polymer or combination of polymers can be used to implement sensors 302, such as any polymer that is solution processable. Examples of polymers can include: poly(vinyl chloride), Poly(styrene-co-maleic anhydride), Poly(styrene-co-maleic acid), partial isobutyl ester, Poly(4-vinylphenol-co-methyl methacrylate), Ethyl cellulose, Poly(caprolactone), Polyvinylpyrrolidone, or Poly(methyl vinyl ether-alt-maleic acid).

In some embodiments, sensors 302, processor 304, and antenna 306 can be mounted to a flexible printed circuit board (FPCB) 308. For example, sensors 302, processor 304, and antenna 306 can be mounted to FPCB 308, and sealed in a flexible coating, such as a soft and flexible transparent coating of poly(dimethylsiloxane) (PDMS). In some embodiments, an aperture(s) can be created in the coating to facilitate exposure of sensors 302 to the environment (e.g., a hole can be cut into the coating, a well can be created during coating in which the sensor can be affixed and/or created, etc.). In some embodiments, a breathable film can be disposed over each of one or more sensors 302. For example, a breathable film can include apertures and/or micropores with a particular size or range of sizes. In such an example, the size of the apertures/micropores can be controlled to tune the amount of vapor that can reach sensors 302. In some embodiments, device 300 can have a relatively small diameter. For example, device 300 can have a diameter in a range of about 20 to 25 millimeters (mm). In some embodiments, a side (e.g., a back side) of FPCB 308 can be coated with an adhesive, which can be used to place the device 300 in any suitable location. For example, device 300 can be bonded to the skin of a subject, to clothing, to a clip that can be affixed to clothing, etc.

In some embodiments, sensors 302 can be any suitable type of chemical sensor and can be implemented using any suitable technique or combination of techniques. For example, sensors 302 can be implemented as multiple, individual, conductive polymer filaments, which can enhance the gas selectivity of a sensor array that includes sensors 302. For example, sensors can be implemented using polymers that reversibly interact with excreted molecules (e.g., excreted via the skin in a subject's sweat, excreted via the respiratory tract in a subject's exhaled breath), which can cause changes in the electrical properties of the individual filaments. In some embodiments, changes in properties of the sensors 302 can be analyzed, and can correspond to the smellprint-signature for a particular chemical or chemicals. For example, device 300 can include sensors 302 configured to passively collect VOCs associated with SARS-CoV-2 infection. In some embodiments, sensors 302 can be configured to respond to a variety of VOCs that are excreted through the skin and associated with health and/or disease.

In some embodiments, each sensor 302 can be relatively small in area (e.g., having an area less than about 5×5 mm² (e.g., an area of about 5×5 mm², and area of about 4×4 mm², and area of about 3×3 mm², an area of about 2×2 mm², etc.). As described below in connection with FIG. 5, each sensor 302 can include polymer filament disposed on an interdigitated comb-type resistor (e.g., mounted on FPCB 308), which can remain uncoated. In some embodiments, implementing sensors 302 using defined areas can facilitate drop-casting of the conductive polymer filaments in two layers to ensure a reliable sensor-manufacturing process. For example, first, multiple-walled carbon nanotubes (MWNTs) can be dissolved in water at a suitable concentration (e.g., at a concentration of 1 percent by weight (wt %)), and deposited onto the interdigitated resistor. The water can be evaporated (e.g., in an oven) such that the MWNTs can form a conductive film. Next, the polymer layer can be dissolved in a suitable solvent at a suitable concentration (e.g., at a concentration of 1 wt %)), and deposited on the conductive film. The solvent can be evaporated (e.g., in an oven), leaving a polymer matrix associated with the conductive film. In some embodiments, the polymer matrices in the array of sensors 302 can be selected based on a linear solvation energy relationship theory to enhance the gas selectivity of the e-nose sensor.

In some embodiments, processor 304 can securely read data from sensors 302 and transmit the data (and/or processed data generated from raw data from sensors 302) to an NFC reader (e.g., NFC reader device 110). In some embodiments, the NFC reader can upload the data and/or processed data to a server for storage and/or analysis (e.g., at regular and/or irregular intervals). In some embodiments, process 304 can be implemented using any suitable device(s) and/or any suitable technique or combination of techniques. For example, processor 304 can be implemented as a system on a chip (SoC) with NFC functionality and an integrated analog-to-digital converter (e.g., a 14-bit analog-to-digital converter), and any other suitable sensors (e.g., a temperature sensor).

In some embodiments, antenna 306 can be implemented using any suitable device(s) and/or any suitable technique or combination of techniques. For example, antenna 306 can be implemented using a magnetic resonant radio frequency (RF) antenna. In such an example, the RF antenna can be tuned to any suitable frequency range to facilitate NFC communication. In some embodiments, antenna 306 and processor 304 can provide a wireless interface for data transmission and power transfer. In some embodiments, device 300 can remain stable under relatively large mechanical bending (e.g., a device having a radius of about 22 mm can be bent up to a radius of about 5 mm without damage).

In some embodiments, implementing device 300 with sensors 302, processor 304, and antenna 306 can facilitate implementation of a wearable e-nose sensor device with a multi-polymer sensor array that is configured to measure VOCs in a battery-free, continuous accumulation mode (e.g., the sensor array can absorb and release chemicals in the air continuously without a power source, and power can be applied to read a measure indicative of resistance associated with the sensors at intervals). To generate a smellprint-signature, an NFC reader device (e.g., computing device 110, such as a smartphone), can be wirelessly coupled to device 300 (e.g., via antenna 306), which can supply power to processor 304. In some embodiments, in response to receiving power, processor 304 can be configured to read the conductivity of filaments in the multi-polymer sensor array.

In some embodiments, device 300 can be placed in a vicinity of a subject's body. For example, as described above, device 300 can be adhered to a portion of a subject's body using a soft skin adhesive interface. In such an example, any suitable adhesive can be used, such as an adhesive gel (e.g., RT Gel 4717, marketed as SILBIONE). As another example, device 300 can be affixed to a subject's clothing or a face mask to capture VOCs from exhaled breath. In such an example, any suitable adhesive can be used to affix device 300, such as a liquid or hot melt film adhesive. In some embodiments, device 300 can be placed on nearly any region of a subject's body, and/or on clothing, jewelry, or a facemask.

In some embodiments, device 300 can be read at any time by bringing an NFC-enabled device (e.g., computing device 110) into relatively close proximity of antenna 306. As described below in connection with FIG. 6, in some embodiments, an NFC-enabled device can be configured to periodically (e.g., at regular and/or irregular intervals) prompt a user to take a reading from device 300 (e.g., via an application installed on computing device 110 that implements a portion of physiological state prediction system 104 and/or a portion of environmental prediction system 114). Additionally, in some embodiments, computing device 110 and/or a server (e.g., server 120) to which computing device 110 uploads data received from device 300 can analyze the data received from device 300 to predict a physiological state of the subject associated with device 300. For example, a classification algorithm can be used to determine a likelihood that the subject is infected with SARS-CoV-2 based on the data from device 300. Note that this is merely an example, and one or more classification algorithms can be trained to predict any suitable physiological state.

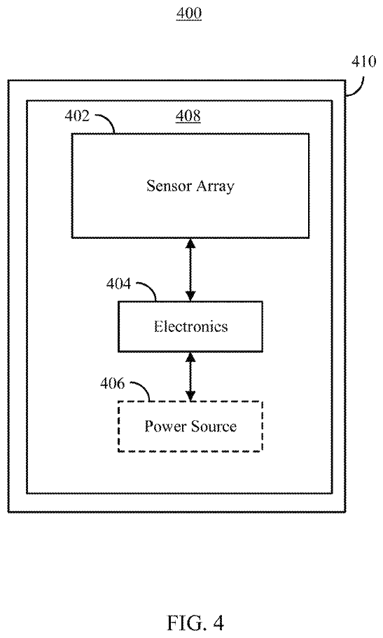

FIG. 4 shows an example 400 of another device with multiple chemical sensors for predicting a physiological state of a subject in accordance with some embodiments of the disclosed subject matter. In some embodiments, sensor device 400 can include a sensor array 402, electronics 404, and a power source 406. In some embodiments, senor array 402, electronics 404, and/or power source 406 can be affixed to one or more substrates 408 (e.g., printed circuit boards, which may or may not be printed circuit boards).

In some embodiments, sensor array 402 can include any suitable chemical sensors or combination of chemical sensors, and/or any other suitable sensors (e.g., a temperature sensor(s), a humidity sensor(s), etc.). For example, sensor array 402 can include sensors similar to sensors 302 described above in connection with FIG. 3.

In some embodiments, electronics 404 can include any suitable components, such as a processor (e.g., processor 222), a communication system(s) (e.g., communication system(s) 226), memory (e.g., memory 228), and/or any other suitable components. In some embodiments, sensor device 404 can be implemented using a system-on-a-chip processor with an integrated WiFi transceiver and analog-to-digital converter(s), which can facilitate real-time wireless transmission of sensor data.

In some embodiments, power source 406 can be implemented using any suitable power source and/or combination of power sources, such as one or more power sources described above in connection with power source 230. For example, sensor device 400 can be implemented using a universal serial bus (USB) type A connector (e.g., USB A 2.0, USB A 3.0). As another example, sensor device 400 can be implemented using a USB type C connector, or any other suitable USB connector. In such examples, sensor device 400 can relatively easily be connected to AC power via an AC/DC wall adapter. As yet another example, sensor device 400 can be implemented using a wall power connector (e.g., a type A wall outlet configured to provide power via a 110 V wall outlet). In such an example, sensor device 400 can include power electronics to convert the wall power received to a suitable DC power.

In some embodiments, components of sensor device 400 can be enclosed within a housing 410, which can include one or more apertures through which air can pass into contact with sensors of sensor array 402. In some embodiments, one or more additional components can be incorporated into sensor device 400 and/or housing 410. For example, a fan can be incorporated into sensor device 400 to circulate air from a surrounding environment through housing 410 and/or past sensor array 402. As another example, a heater can be incorporated into sensor device 400, which can increase a speed at which sensors of sensor array 402 release VOCs. As yet another example, an occupancy sensor (e.g., a camera, an infrared occupancy sensor, etc.) can be incorporated into sensor device 400, which can be used to record when a room/area is occupied. As still another example, one or more environmental sensors (e.g., audio, humidity, air pressure, illumination, motion, etc.) can be incorporated into sensor device 400, and can be used to record environmental conditions.

In some embodiments, sensing device 400 can be used in lieu of, or in addition to, a portable sensing device (e.g., sensing device 300). While direct sensing (e.g., using sensing device 300) can produce relatively high signal quality (e.g., potentially higher than sensing device 400), such direct sensing typically requires active user data collection (e.g., as described above in connection with FIG. 3) or batteries with a limited capacity (e.g., which then must be periodically recharged and/or replaced to continue using the device). Similar diagnostic information can be captured by an indirect e-nose sensor (e.g., sensor device 400) that is not physically attached to the subject. Such an indirect sensing approach can facilitate greater flexibility in size and/or placement, allowing the sensor to be integrated into the environment (e.g., located near a wall power outlet), eliminating the need for batteries or active data collection. Additionally, using a power source such as wall power may facilitate more frequent sensing (e.g., compared to readings performed when a user brings an NFC device into proximity of sensing device 300). For example, data can be collected using a powered device on the ordered of seconds, or minutes, rather than hours. In a more particular example, a smellprint-signature of an environment around sensing device 400 (e.g., a subject's room) can be collected once per minute, and the data can be uploaded to secure cloud-based storage (e.g., implemented by server 120) before and/or after processing (e.g., as raw data and/or processed data).

FIGS. 5A and 5B show examples of a chemical sensor that can be used to implement a device with one or more chemical sensors for predicting a physiological state of a subject in accordance with some embodiments of the disclosed subject matter. As shown in FIG. 5A, in some embodiments, a chemical sensor can be implemented using a copper pad with interdigitated copper conductor pad through which a signal can be passed, and a polymer mat with inclusions of carbon nanotubes (e.g., as described above in connection with FIG. 3). As the polymer filaments absorb one or more chemicals, the electrical properties of the polymer changes, changing the electrical properties of the mat (e.g., changing the resistance and dielectric permittivity of the mat).

As shown in FIG. 5B, an assembled chemical sensor can include a polymer mat disposed on a copper pad (e.g., with interdigitated conductors), which can be electrically coupled to a processing device (e.g., processor 222).

FIG. 6 shows an example 600 of a process for predicting a physiological state of a subject in accordance with some embodiments of the disclosed subject matter. At 602, process 600 can include calibrating a device with one or more chemical sensors and/or recording one or more baseline sensor outputs. For example, in some embodiments, calibration can include exposing the sensor(s) to known concentrations of one or more chemicals (e.g., VOCs), and recording readings from the sensor(s) after a particular time(s) have elapsed. As another example, process 600 can include recording readings from the sensor(s) when the sensor is in an ambient environment (e.g., a hospital room) and/or in a controlled environment (e.g., in a vacuum, in an environment with particular concentration of one or more chemicals). In some embodiments, readings recorded at 602 can be stored, and can be used as a basis for comparison to readings when the chemical sensor is in use.

At 604, process 600 can communicate with a mobile NFC reader device that has been brought into proximity of the sensor device. In some embodiments, a device executing at least a portion of process 600 can receive power from an NFC reader device brought into proximity of an antenna (e.g., an RF antenna), and can attempt to establish a communication channel with the NFC reader device (e.g., in response to receiving power). In some embodiments, 604 can be omitted. For example, if a powered device is executing at least a portion of process 600, process 600 can omit 604 and can record readings (e.g., at 606) regardless of whether a reader device is brought into proximity to an antenna.

In some embodiments, a mobile NFC reader device (e.g., computing device 110) can prompt a user to bring the mobile NFC reader device into proximity of the sensor device at regular and/or irregular intervals (e.g., every hour, every two hours, etc.).

In some embodiments, the sensor device can be worn in various locations. For example, a sensor device can be worn on the skin (e.g., torso or upper limbs). As another example, a sensor device can be clipped to the anterior top of a hospital gown. As yet another example, a sensor device can be clipped to the inside of the waist of an undergarment. As still another example, a sensor device can be integrated into a mask (e.g., a cloth face mask). In such an example, a subject can be instructed to wear the mask for at least 10 minutes prior to collecting data. As a further example, a sensor device can be placed on a bedside table.

At 606, process 600 can record sensor outputs from the one or more chemical sensors at a particular point in time (e.g., time t). For example, process 600 can record sensor outputs in response to a mobile NFC reader powering a processor of a device executing process 600 (e.g., sensor device 300). As another example, process 600 can record sensor outputs in response to a predetermined period of time having elapsed (e.g., one second, 30 seconds, one minute, two minutes, five minutes, etc.).

In some embodiments, process 600 can record a single value associated with each sensor at a particular point in time. For example, process 600 can record a resistance value, a conductance value, a capacitance value, and/or any other suitable value associated with each sensor at 606.

As described above in connection with FIGS. 2 to 4, an e-nose sensor can detect VOCs based on reversible interactions that alter the electrical properties (e.g., resistance/conductance, capacitance) of the individual polymer filaments. For example, resistance/conductance can be measured using a DC current. In some embodiments, such sensors can be configured to identify individual compounds or mixtures with a relatively high degree of specificity and sensitivity. A smellprint-signature can include conductivity values (and/or any other suitable values) of each of the polymer filaments in the sensor array.

At 608, process 600 can generate a profile indicative of a physiological state of one or more subjects and/or presence of potential environmental threats based on sensor outputs at a particular point in time (e.g., at time t). In some embodiments, process 600 can use any suitable technique or combination of techniques to generate the profile indicative of the physiological state of the one or more subjects. For example, process 600 can use values from one or more sensors to generate a profile (e.g., a smellprint).

As another example, one or more dimensionality reduction techniques can be used to reduce the dimensionality of the data. In a more particular example, a principal components analysis (PCA) can be carried out on the data to reduce the dimensionality of the data.

In some embodiments, data used to generate a profile can be relative data (e.g., rather than raw data). For example, a reading from a first sensor can be compared to an initial background reading and/or calibration reading, and a value used to generate a profile can be the relative change in the value (e.g., in lieu of the raw value).

At 610, process 600 can record output from one or more additional sensors (e.g., sensors other than chemical sensors for which data was recorded at 606, such as physiological sensors). In some embodiments, process 600 can record output from any other suitable sensor(s). For example, process 600 can record heart rate data, blood oxygen data, leg movement data, activity data (e.g., indicating a level of one or more activities and/or general movement), electrocardiogram (ECG) data, electroencephalogram data, blood pressure data, etc. In some embodiments, 610 can be omitted, for example, when a chemical sensor profile (e.g., generated at 608) can be used without additional input.

In some embodiments, after data collection from a sensor device(s) (e.g., at 606 and/or 610), data can be transmitted in real-time to a secure server for storage (e.g., cloud-based storage). For example, a computing device (e.g., computing device 110) can collect data from a sensor device (e.g., sensor device 300) using an application installed on the device (e.g., implemented as part of physiological state prediction system 104 and/or as part of environmental prediction system 114). In some embodiments, if a network connection is unavailable (e.g., if cell service and/or WiFi is unavailable), data can be stored on the computing device for batch upload at a later time.

At 612, process 600 can predict a physiological state of one or more subjects based on the profile generated at 608 and, optionally, signals from one or more other sensors recorded at 610. In some embodiments, process 600 can use any suitable technique or combination of technique to predict a physiological state of the one or more subjects. For example, process 600 can use a trained machine learning model to predict a physiological state of the subject.

In some embodiments, process 600 can use a trained classification model to distinguish between subjects with different physiological states (e.g., subjects infected with SARS-CoV-2 and uninfected subjects, subjects that are likely to experience disease caused by C. diff infection and subjects not infected with C. diff and/or subjects that are unlikely to experience diseases caused by C. diff despite the presence of C. diff, subjects that do or do not have cystic fibrosis, subjects that do or do not have asthma, subjects that do or do not have chronic obstructive pulmonary disease, etc.). In such embodiments, a machine learning model can be trained using one or more supervised learning techniques using smellprint-signature data to identify any suitable physiological state (e.g., based on data recorded from samples confirmed to represent SARS-CoV-2 infection with a PCR test result used to label the data, based on data recorded from samples confirmed to represent C. diff infection causing diarrhea). In some embodiments, any suitable data can be used to train the machine learning model. For example, conductivity (and/or any other suitable value indicative of an electrical property of the sensor, such as capacitance) of each of the polymer filaments of a sensor device can be extracted as features from the sensor, and a two-dimensional principal component analysis (PCA) can be performed to reduce the dimensionality of the features.

In some embodiments, any suitable machine learning model can be trained to predict a physiological state of a user among two or more physiological states. For example, the machine learning model can trained using a k-nearest neighbors technique. As another example, the machine learning model can be implemented using a trained support vector machine. As yet another example, the machine learning model can be implemented using a random forest model. As still another example, the machine learning model can be implemented using a trained artificial neural network. In some embodiments, cross-validation can be used to determine the accuracy of the predictive model. Note that these are merely examples, and other examples of other techniques that can be used to predict a physiological state of a user can include k-means clustering, spatial clustering, expectation-maximization, clustering using Gaussian mixture models, etc.

In some embodiments, a machine learning model(s) can be trained based on a pilot study. For example, a machine learning model can be trained to predict SARS-CoV-2 infection based on data from 60 subjects enrolled in a pilot study, including 30 with confirmed SARS-CoV-2 infection and 30 controls (e.g., confirmed negative for SARS-CoV-2 infection). The controls can be patients admitted to the hospital that do not have confirmed SARS-CoV-2 infection (and/or that have received a negative test result for SARS-CoV-2 infection).

Additionally or alternatively, at 612, process 600 can predict presence of one or more environmental threats based on the profile generated at 608 and, optionally, signals from one or more other sensors recorded at 610. In some embodiments, process 600 can use any suitable technique or combination of technique to predict presence of an environmental threat. For example, process 600 can use a trained machine learning model to predict presence of an environmental threat using a profile generated at 608. In some embodiments, at 612, process 600 can predict presence of any suitable potential environmental threat. For example, process 600 can predict the presence of potential chemical threats such as poisonous vapors, aerosols, or liquids that have toxic effects on people, animals, and/or plants. As a more particular example, process 600 can predict the presence of industrial chemicals (e.g., ammonia, chlorine, nitric acid, sulfur dioxide, etc.), environmental contaminants (e.g., pesticides, sulfides, nitrates, petrochemicals, etc.), food contaminants, explosive vapors, disease biomarkers, and/or other chemical vapors (e.g., benzene, toluene, ethyl benzene, xylene, gasoline, etc.).

As another example, process 600 can predict the presence of environmental pollutants (e.g., for air quality monitoring). As a more particular example, process 600 can predict the presence of chemicals such as carbon monoxide, isobuthane, methane, ethanol, nitrous oxide, hydrogen sulfide, acetone, chloroform, enzene, toluene, ethyl alcohol, methyl alcohol, ethyl benzene, xylenes, etc. In such examples, process 600 can be used for odor impact assessments, control applications, and/or any other suitable uses related to environmental pollutants.

As yet another example, process 600 can predict the presence of potential environmental threats (e.g., chemical threats, biological threats, infectious diseases, environmental pollution, etc.) that cannot be directly detected by the chemical sensors based on the response of a subject to the potential environmental threat. In such an example, process 600 can predict the presence of an environmental threat based on detection of VOCs excreted by the subject. It is currently not possible to directly detect the presence of many biological threats and/or infectious diseases using an electronic nose. In some embodiments, process 600 can be used to indirectly detect the presence of otherwise undetectable threats by monitoring VOCs and/or change in VOCs that are excreted by the human body. Such VOC compounds excreted may be end products of human metabolism and can be indicative of health and/or disease. VOCs may be absorbed by a person through breathing, ingestion, and/or through the skin. Additionally, VOCs may also be generated by bacteria, which are present in the human intestine or other body parts. Both bacteria that are normally present and bacteria that are present abnormally may generate VOCs. In addition, VOCs excreted by a person can result from human metabolism of molecules absorbed as described above. In some embodiments, process 600 can use a classification model trained predict presence of particular biological threats based on a specific pattern(s) for a known threat/challenge, and/or based on a change from nominal that may indicate the presence of a novel threat/challenge. In some embodiments, such techniques (e.g., based on VOCs emitted by a human body in response to an interaction with an environmental threat) can be used to predict presence of chemical threats, environmental pollution, and/or a variety of infectious and non-infectious diseases.

In some embodiments, process 600 can use a trained classification model to distinguish between different environmental threats (e.g., the presence of a particular chemical or biological threat). In such embodiments, a machine learning model can be trained using one or more supervised learning techniques using smellprint-signature data to identify any suitable environmental threat that can be detected using the chemical sensors (e.g., based on data recorded from outputs generated by chemical sensors that have been exposed to the environmental threat). In some embodiments, any suitable data can be used to train the machine learning model. For example, conductivity (and/or any other suitable value indicative of an electrical property of the sensor, such as capacitance) of each of the polymer filaments of a sensor device can be extracted as features from the sensor, and a two-dimensional principal component analysis (PCA) can be performed to reduce the dimensionality of the features.

In some embodiments, any suitable machine learning model can be trained to predict presence of various environmental threats. For example, the machine learning model can trained using a k-nearest neighbors technique. As another example, the machine learning model can be implemented using a trained support vector machine. As yet another example, the machine learning model can be implemented using a random forest model. As still another example, the machine learning model can be implemented using a trained artificial neural network. In some embodiments, cross-validation can be used to determine the accuracy of the predictive model. Note that these are merely examples, and other examples of other techniques that can be used to predict a physiological state of a user can include k-means clustering, spatial clustering, expectation-maximization, clustering using Gaussian mixture models, etc.

At 614, process 600 can cause a predicted physiological state of the one or more subjects to be presented to a user (e.g., a user of computing device 110). Additionally or alternatively, at 614, process 600 can cause a predicted presence of an environmental threat to be presented to a user (e.g., a user of computing device 110).

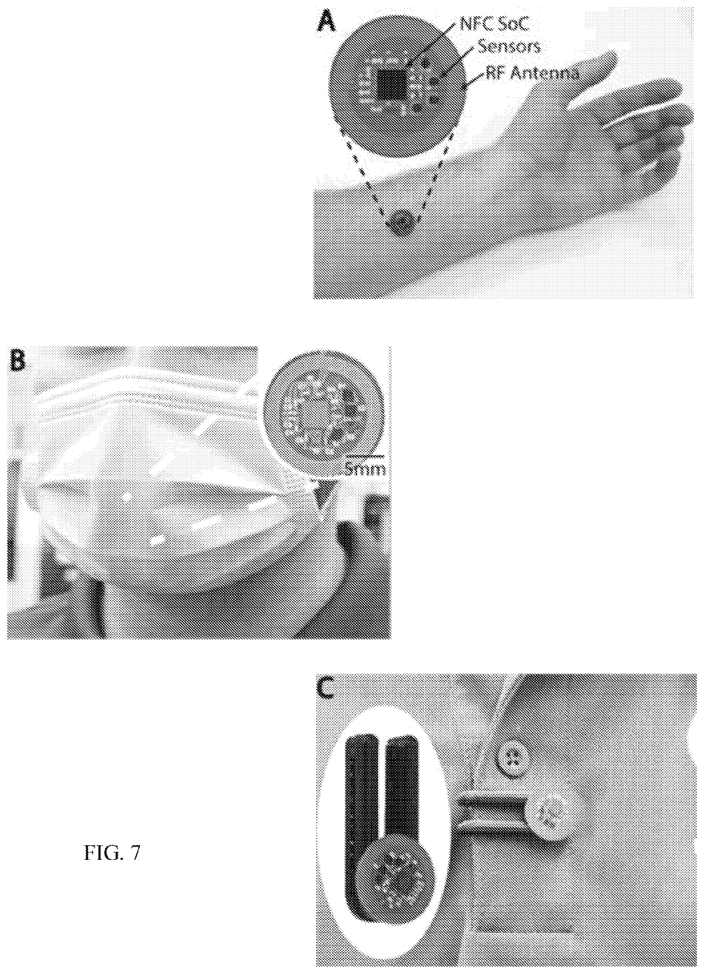

FIG. 7 shows examples of techniques for configuring a device with multiple chemical sensors for predicting a physiological state of a subject as a wearable device in accordance with some embodiments of the disclosed subject matter. As shown in FIG. 7, panel A, a sensor device (e.g., sensor device 300) can be affixed to a subject's skin (e.g., on an arm, torso, neck, etc.). As shown in FIG. 7, panel B, a sensor device (e.g., sensor device 300) can be affixed to a mask to be worn by a subject (e.g., on a surface of the mask facing the subject's mouth and/or nose). As shown in FIG. 7, panel C, a sensor device (e.g., sensor device 300) can be affixed to a fastener (e.g., a clip), which can be used to mount the sensor device on an article of clothing worn by a subject.

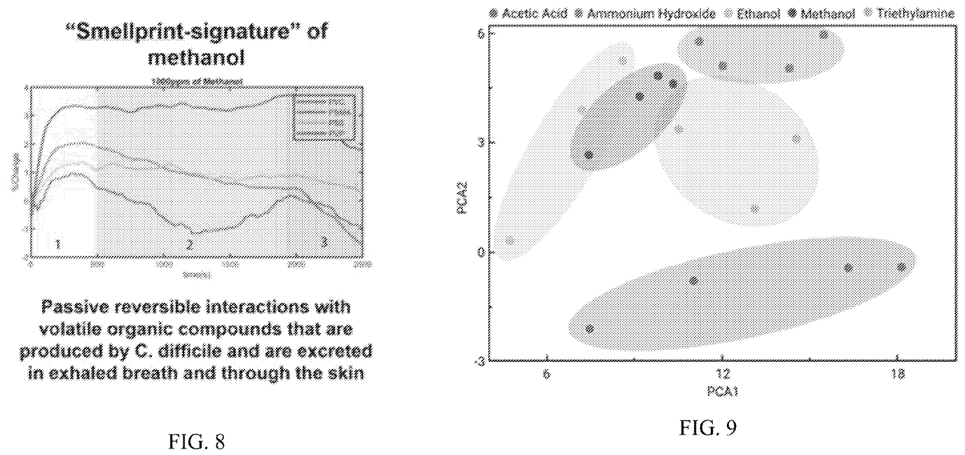

FIG. 8 shows an example of relative output of four different chemical sensors over time when exposed to a known concentration of methanol. As shown in FIG. 8, sensors implemented with different polymers can react differently to a particular compound. The sensors used to generate the measurements in FIG. 8 include a sensor implemented using polyvinal chloride (PVC), a sensor implemented using Poly(styrene-co-maleic anhydride) (PSMA), a sensor implemented using poly(styrene-co-maleic acid) partial isobutyl/methyl mixed ester, and a sensor implemented using polyvinylpyrrolidone (PVP).

FIG. 9 shows an example of results of a principal component analysis (PCA) of outputs of the four different chemical sensors used to produce the example of FIG. 8 when analyzed at after exposure to a various volatile organic chemicals. As shown in FIG. 9, results of PCA can generally cluster relatively close together. In some embodiments, a sensor device (e.g., sensor device 300, sensor device 400) can be tested in a controlled setting with six off-the-shelf VOCs (acetic acid, ammonium hydroxide, ethanol, methanol, pyridine, and trithylamine), and the results can be used to evaluate sensor performance. The smellprint-signature for each VOC cam be recorded (e.g., in a random order) a predetermined number of times for each VOC (e.g., 10 times each), resulting in a set of smellprint-signatures (e.g., 60 smellprint-signatures). PCA can be performed to reduce the dimensionality of the features and cross-validation can be used to determine the accuracy of a predictive model developed to determine the presence of different VOCs.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, any other suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the processes of FIG. 6 can be executed or performed in any order or sequence not limited to the order and sequence shown and described in the figures. Also, some of the above steps of the processes of FIG. 6 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow Features of the disclosed embodiments can be combined and rearranged in various ways.

What is claimed is:

1. A system for predicting presence a physiological state of a subject, the system comprising:
a sensor device comprising:
a plurality of chemical sensors,
wherein the plurality of chemical sensors comprise polymer-based sensor or a metal-oxide-based sensor;
a transmitter; and
a processor coupled to the plurality of chemical sensors and the transmitter, the processor programmed to:
measure, for each of the plurality of sensors, a respective value of a plurality of values,
wherein each of the plurality of values is a conductance value or each of the plurality of values is a capacitance value; and
transmit, via the transmitter, a profile based on the plurality of values to a computing device; and
a computing device comprising:
a processor programmed to:
receive the profile; and
predict a physiological state of a subject based on the profile,
wherein the physiological state of the subject comprises:
whether the subject is infected with SARS-CoV-2 virus; or
whether the subject is likely to experience disease caused by Clostridioides difficile.

2. A system for predicting presence of one or more molecules using one or more chemical sensors, the system comprising:
a plurality of chemical sensors;
a transmitter; and
a processor coupled to the plurality of chemical sensors and the transmitter, the processor programmed to:
measure, for each of the plurality of sensors, a respective value of a plurality of values; and
transmit, via the transmitter, a profile based on the plurality of values to a computing device.

3. The system of claim 2, wherein the transmitter comprises a radio-frequency antenna.

4. The system of claim 3, wherein the processor is further programmed to:
transmit the profile via near-field communication.

5. The system of claim 4, wherein the processor is configured to receive power via the radio-frequency antenna.

6. The system of claim 2, wherein the processor is further programmed to:
perform a principal component analysis based on the plurality of values.

7. The system of claim 6, wherein the processor is further programmed to:
generate the profile based on results of the principal component analysis.

8. The system of claim 6, wherein the profile comprises results of the principal component analysis.

9. The system of claim 6, wherein the processor is further programmed to:
predict a physiological state of a subject based on results of the principal components analysis; and
generate the profile based on the predicted physiological state of the subject.

10. The system of claim 2, wherein the system further comprises a power source electronically coupled to the processor.

11. The system of claim 10, wherein the power source comprises a battery.

12. The system of claim 2, wherein each of the plurality of values is a conductance value.

13. The system of claim 2, wherein each of the plurality of values is a capacitance value.

14. The system of claim 2, wherein the physiological state is indicative of whether the subject is infected with SARS-CoV-2 virus.

15. The system of claim 2, wherein the plurality of chemical sensors comprises a polymer-based sensor.

16. The system of claim 2, wherein the plurality of chemical sensors comprises a metal-oxide-based sensor.

17. A system for predicting a physiological state of a subject using one or more chemical sensors, the system comprising:
a processor programmed to:
receive a profile based on a plurality of values generated by a sensor device comprising a plurality of chemical sensors;
predict a physiological state of a subject based on the profile.

18. The system of claim 17, wherein the system comprises a server, and wherein the profile is received from the sensor device via a mobile computing device.

19. The system of claim 17, wherein the system comprises a server, and wherein the profile is received from the sensor device via a wide area network.

20. The system of claim 17, wherein the system comprises a radio-frequency antenna.

21. The system of claim 20, wherein the processor is further programmed to:
receive the profile via near-field communication.

22. The system of claim 20, wherein the processor is configured to provide power to the sensor device via the radio-frequency antenna.

23. The system of claim 17, wherein the processor is further programmed to:
predict a physiological state of a subject based on results of a principal components analysis of the plurality of values; and
generate the profile based on the predicted physiological state of the subject.

24. The system of claim 23, wherein the processor is further programmed to:
perform the principal component analysis based on the plurality of values.

25. The system of claim 23, wherein the profile is based on results of the principal component analysis.

26. The system of claim 17, wherein each of the plurality of values is a conductance value.

27. The system of claim 17, wherein each of the plurality of values is a capacitance value.

28. The system of claim 17, wherein the physiological state is indicative of whether the subject is infected with SARS-CoV-2 virus.

29. The system of claim 17, wherein the plurality of sensors comprises a polymer-based sensor.

30. The system of claim 17, wherein the plurality of sensors comprises a metal-oxide-based sensor.

31. The system of claim 17, wherein the processor is further programmed to:
receive physiological data generated by a physiological sensor; and
predict the physiological state of the subject based on the profile and the physiological data received from the physiological sensor.

32. The system of claim 31, wherein the processor is further programmed to:
receive the physiological data from the sensor device.

33. A system for predicting presence of one or more environmental threats using one or more chemical sensors, the system comprising:
a processor programmed to:
receive a profile based on a plurality of values generated by a sensor device comprising a plurality of chemical sensors;
predict presence of one or more environmental threats based on the profile.

34. The system of claim 33, wherein the system comprises a server, and wherein the profile is received from the sensor device via a mobile computing device.

35. The system of claim 33, wherein the system comprises a server, and wherein the profile is received from the sensor device via a wide area network.

36. The system of claim 33, wherein the system comprises a radio-frequency antenna.

37. The system of claim 36, wherein the processor is further programmed to:
receive the profile via near-field communication.

38. The system of claim 36, wherein the processor is configured to provide power to the sensor device via the radio-frequency antenna.

39. The system of claim 33, wherein the processor is further programmed to:
predict presence of an environmental threat based on results of a principal components analysis of the plurality of values; and
generate the profile based on the predicted physiological state of the subject.

40. The system of claim 39, wherein the processor is further programmed to:
perform the principal component analysis based on the plurality of values.

41. The system of claim 39, wherein the profile is based on results of the principal component analysis.

42. The system of claim 33, wherein each of the plurality of values is a conductance value.

43. The system of claim 33, wherein each of the plurality of values is a capacitance value.

44. The system of claim 33, wherein the environmental threat is indicative of whether the sensor device has been exposed to one or more of: ammonia, chlorine, nitric acid, sulfur dioxide, carbon monoxide, isobutane, methane, ethanol, nitrous oxide, hydrogen sulfide, acetone, chloroform, benzene, toluene, ethyl alcohol, methyl alcohol, ethyl benzene, xylene, benzene, toluene, or gasoline.

45. The system of claim 33, wherein the plurality of sensors comprises a polymer-based sensor.

46. The system of claim 33, wherein the plurality of sensors comprises a metal-oxide-based sensor.
